(19) United States
(12) Patent Application Publication
(10) Pub. No.: US 2023/0334763 A1
(43) Pub. Date: Oct. 19, 2023

(54) CREATING COMPOSITE DRAWINGS USING NATURAL LANGUAGE UNDERSTANDING

(71) Applicant: SYNTHESIS HEALTH INC., Vancouver (CA)

(72) Inventor: Murray Aaron Reicher, Rancho Santa Fe, CA (US)

(21) Appl. No.: 18/302,563

(22) Filed: Apr. 18, 2023

Related U.S. Application Data
(60) Provisional application No. 63/332,491, filed on Apr. 19, 2022.

(51) Int. Cl. G06T 15/08; G06T 15/40; G06F 40/279; G16H 30/20; G16H 30/40; G16H 15/00

(52) U.S. Cl. CPC G06T 15/08; G06T 15/40; G06F 40/279; G16H 30/20; G16H 30/40; G16H 15/00; G06T 2210/41; G06T 2210/32

(57) ABSTRACT

Natural language processing of a physician's comments regarding a medical image may be executed by artificial intelligence software to determine a state (e.g., normal or abnormal) of various anatomical features (e.g., ligaments, tendons, bones, muscles, etc.). The determined anatomical features and their corresponding states may then be used to select one or more representative medical images from a library of stored images (e.g., illustrations or photographs). This process may be repeated to identify multiple representative medical images for different anatomical features and states, and the multiple medical images may be combined (such as by morphing, overlaying, or otherwise combining the images) to form a composite image that illustrates the specific patient anatomy.

### BACKGROUND

When a reading physician (or other user) interprets a medical imaging exam, for example a shoulder MRI, he or she describes the findings and conclusion in a clinical report. The report can be created using a report template that pre-populates certain default findings and/or organizes the report into an itemized list of anatomically based categories to be reported. In some instances, certain images (e.g., particular slices or portions of an MRI three-dimensional volume) that exemplify the key findings are annotated by the reading physician and may be added to the text report or hyperlinked to the appropriate text in the report.

### SUMMARY

The reading physician often inspects a large number of images (hundreds or even thousands) in an exam and then uses text and occasionally images to communicate to the referring doctor and patient. The report may then be reviewed and interpreted by others (e.g., the referring doctor, patient, insurance representative, etc.) and the picture that forms in each of those individuals minds about the patient anatomy may substantially differ from what the reading physician had envisioned. (and/or intended to convey). This may lead to diagnostic and treatment errors.

The following description discusses various processes and components that may perform artificial intelligence (“AI”) processing or functionality. AI generally refers to the field of creating computer systems that can perform tasks that typically require human intelligence. This includes understanding natural language, recognizing objects in images, making decisions, and solving complex problems. AI systems can be built using various techniques, like neural networks, rule-based systems, or decision trees, for example. Neural networks learn from vast amounts of data and can improve their performance over time. Neural networks may be particularly effective in tasks that involve pattern recognition, such as image recognition, speech recognition, or Natural Language Processing.

Natural Language Processing (NLP) is an area of artificial intelligence (AI) that focuses on teaching computers to understand, interpret, and generate human language. By combining techniques from computer science, machine learning, and/or linguistics, NLP allows for more intuitive and user-friendly communication with computers. NLP may perform a variety of functions, such as sentiment analysis, which determines the emotional tone of text; machine translation, which automatically translates text from one language or format to another; entity recognition, which identifies and categorizes things like people, organizations, or locations within text; text summarization, which creates a summary of a piece of text; speech recognition, which converts spoken language into written text; question-answering, which provides accurate and relevant answers to user queries, and/or other related functions. Natural Language Understanding (NLU), as used herein, is a type of NLP that focuses on the comprehension aspect of human language. NLU may attempt to better understand the meaning and context of the text, including idioms, metaphors, and other linguistic nuances. As used herein, references to specific implementations of AI, NLP, or NLU should be interpreted to include any other implementations, including any of those discussed above.

As discussed herein, natural language processing of a physician's comments regarding a medical image may be executed by artificial intelligence (“AI”) software (e.g., one or more neural network, reinforcement learning algorithm, Bayesian network, evolutionary algorithm, etc.) to determine a state (e.g., normal or abnormal) of various anatomical features (e.g., ligaments, tendons, bones, muscles, etc.). The determined anatomical features and their corresponding states (e.g., a subscapularis muscle, abnormal or teres minor, normal) may then be used to select one or more representative medical images from a library of stored images (e.g., illustrations or photographs). This process may be repeated to identify multiple representative medical images for different anatomical features and states, and the multiple medical images may be combined (such as by morphing, overlaying, or otherwise combining the images) to form a composite image that illustrates the specific patient anatomy.

In one example implementation, a neural network may be trained to understand language related to normal and abnormal (e.g., pathological) findings in an applicable region of interest. A library of images demonstrating normal and various specific abnormal findings may be indexed (or otherwise categorized) by respective language meanings and/or body parts. The computer device may then receive the language description, select the proper image(s) and generate one or more composite images based on the images that best match the descriptions. Language understanding may be used to select and alter the images, such as to depict the size of location of finding, to reflect the age/gender of the patient, or to reflect a classification system related to normal or abnormal anatomy. The selection and alteration of the image components may be aided by other techniques using artificial intelligence, such as registering one or more components to anatomical structures on medical images of the patient or reference images, and/or altering and/or selecting one or more composite components by automatically comparing the patient's medical images to the available components. The illustrative images may be included in the report and/or may be stored with the exam images, or both. They may be single images or an illustrative volume of image data may be generated that can be further processed using multiplanar reformatting or volume rendering techniques, for example. By starting with a library of composite images, the system may be more accurate in creating the final images.

A system of one or more computers can be configured to perform the below example operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Example 1. A computing system comprising: a hardware computer processor; a non-transitory computer readable medium having software instructions stored thereon, the software instructions executable by the hardware computer processor to cause the computing system to perform operations comprising: access a stored library of illustrations of various patient anatomy; display one or more medical images; receive, from a viewer of the one or more medical images, a description of the one or more images; select, based on natural language understanding of the description, one or more illustrations in the stored library; and generate a composite image based on the selected one or more illustrations associated with the description.

Example 2. The computing system of Example 1, wherein the description of the one or more images is in a medical imaging report.

Example 3. The computing system of Example 1, wherein the description of the one or more images is received via input from the user of the computing system.

Example 4. The computing system of Example 1, wherein the illustrations are indexed based on one or more of an imaging exam type or a report template.

Example 5. The computing system of Example 1, wherein the operations further comprise: creating a matching DICOM frame of reference between the one or more medical images and the generated composite image.

Example 6. The computing system of Example 1, wherein the composite image is a volumetric composite image.

Example 7. The computing system of Example 6, wherein the operations further comprise: receiving user input requesting reformatting of the volumetric composite image into a three-dimensional or multiplanar images; and performing the requested reformatting.

Example 8. The computing system of Example 1, wherein the operations further comprise: receiving user input selecting a first of the illustrations in the composite image; and regenerating the composite image without the first of the illustrations, wherein positions of one or more other illustrations become visible in the regenerated composite image.

Example 9. The computing system of Example 1, wherein the operations further comprise: receiving user input selecting an anatomical feature; determining one or more portions of the composite image overlapping or obscuring view of the anatomical feature in the composite image; and regenerating the composite image to at least partially remove the one or more portions of the composite image overlapping or obscuring view of the anatomical feature.

Example 10. The computing system of Example 9, wherein the user input is received by user selection of text in a medical imaging report.

Example 11. The computing system of Example 1, wherein the operations further comprise: determining a report description associated with the one or more selected illustrations; and generating at least portions of a report based on the determined report descriptions.

Example 12. A computerized method, performed by a computing system having one or more hardware computer processors and one or more non-transitory computer readable storage device storing software instructions executable by the computing system to perform the computerized method comprising: (a) determining an anatomical area of a patient; (b) identifying an image of the anatomical area from a stored library of images of various patient anatomy; (c) displaying the image; (d) receiving, from a viewer, a description of a feature of the patient; (e) determining, based on application of natural language understanding to the description, a characteristic of the patient anatomy; (f) identifying a feature image in the stored library that is associated with the determined characteristic of the patient anatomy; (g) generating a composite image based on the image and the feature image; (h) replacing display of the image with the composite image; and (i) repeating actions (d) through (h) until no further features of the patient are described by the viewer.

Example 13. The method of Example 12, wherein the characteristic of the patient anatomy indicates whether an anatomical feature is normal or abnormal.

Example 14. The method of Example 12, wherein the characteristic of the patient anatomy indicates a quantitative characteristic of the patient anatomy.

Example 15. The method of Example 12, wherein each of the images in the stored library is associated with metadata indicating characteristics of the image.

Example 16. The method of Example 15, wherein the metadata includes an indication of anatomical area and characteristic of the anatomical area depicted in the corresponding image.

Example 17. The method of Example 12, wherein the image is a photograph of the patient.

Example 18. The method of Example 12, wherein the feature image is an illustration.

Example 19. The method of Example 12, wherein said generating the composite image is performed by a generative artificial intelligence model.

Example 20. The method of Example 12, further comprising: updating the composite image based on an age, gender, height, or weight of the patient.

Example 21. The method of Example 12, wherein the composite image is an animated image.

Example 22. The method of Example 12, wherein the composite image is interactable based on user inputs.

Example 23. The method of Example 22, wherein the interactions include one or more of adjusting rotation, adjusting magnification, expanding or contracting an area of anatomy depicted.

Example 24. The method of Example 12, wherein the composite image is a three-dimensional image.

Example 25. The method of Example 12, wherein each of the feature images is a photograph, line art drawing, sketch, digital painting, 3D rendering, icon, CAD drawing, or realistic drawing.

Example 26. The method of Example 12, wherein actions (e) through (h) are performed in substantially real-time as the corresponding description of the feature of the patient is received from the viewer.

Example 27. The method of Example 12, wherein the description of the feature of the patient is extracted from a report.

Example 28. The method of Example 12, wherein the description of the feature of the patient is received via voice input from the user.

Example 29. A computerized method, performed by a computing system having one or more hardware computer processors and one or more non-transitory computer readable storage device storing software instructions executable by the computing system to perform the computerized method comprising: determining an anatomical area of a patient; parsing a report to identify a plurality of descriptions of the patient; for each of the identified descriptions in the report: determining a corresponding anatomical feature; determining, based on application of natural language understanding to the description, a state of the anatomical feature, wherein the state is either a normal state or an abnormal state; selecting a feature image in a stored library of images that is associated with the determined anatomical feature having the determined state; generating a composite image based on each of the selected feature images; and displaying the composite image.

Example 30. The computerized method of Example 29, wherein each anatomical feature is one or more ligament, muscle, or bone.

Example 31. The computerized method of Example 29, further comprising: morphing the composite image to match one or more patient characteristics.

Example 32. The computerized method of Example 31, wherein the patient characteristics are determined based on analysis of one or more photographs or medical images of the patient.

### BRIEF DESCRIPTION OF THE DRAWINGS

FIG. 1 illustrates an example computing system (also referred to herein as a “computing device” or “system”).

FIGS. 2A-2D illustrate examples of different types of images that are each representative of a same anatomic structure, each with different visual characteristics and variations in level of detail.

FIG. 3A illustrates several example images of different anatomical features associated with shoulders.

FIG. 3B illustrates an example of images showing a front and back view of bones associated with a shoulder.

FIG. 3C illustrates an example composite image that is generated by selection of images depicting anatomical features from an image library.

FIG. 3D illustrates an example composite image of muscles and some tendons.

FIG. 3E is an example composite image showing combinations of anatomical features of various types (e.g., bones, ligaments, muscles) all combined into a composite medical image.

FIGS. 3F-3I each illustrate a composite medical image, each with different features images associated with different states of the supraspinatus tendon and/or supraspinatus muscle included in the composite medical images.

FIG. 4 is an example imaging report that may be processed to generate a composite image.

FIG. 5 is a flowchart illustrating one example of a method that may be performed to generate a composite image based on one or more images matching descriptions of patient conditions.

### DETAILED DESCRIPTION

Embodiments of the invention will now be described with reference to the accompanying figures, wherein like numerals refer to like elements throughout. The terminology used in the description presented herein is not intended to be interpreted in any limited or restrictive manner, simply because it is being utilized in conjunction with certain specific embodiments. Furthermore, embodiments of the invention may include several novel features, no single one of which is solely responsible for its desirable attributes or which is essential to practicing the inventions herein described.

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The systems and methods discussed herein may be performed by various computing systems, which are referring to herein generally as a computing system.

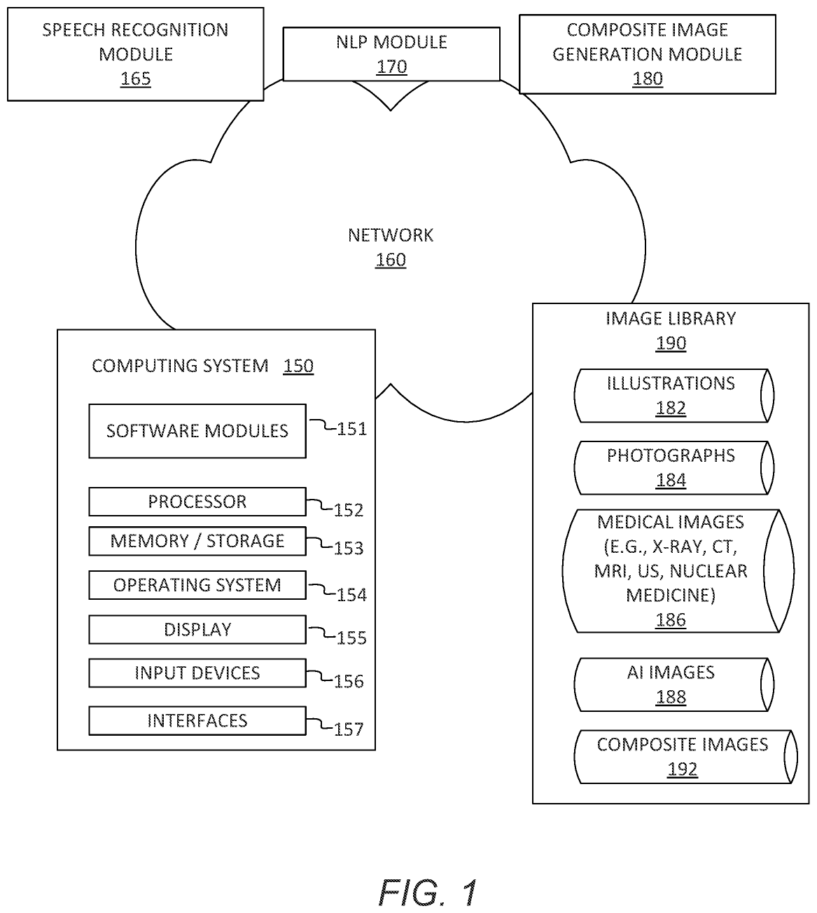

FIG. 1 illustrates an example computing system 150 (also referred to herein as a “computing device 150” or “system 150”). The computing system 150 may take various forms. In one embodiment, the computing system 150 may be a computer workstation having modules 151, such as software, firmware, and/or hardware modules. In other embodiments, modules 151 may reside on another computing device, such as a web server, and the user directly interacts with a second computing device that is connected to the web server via a computer network.

In various embodiments, the computing system 150 comprises one or more of a server, a desktop computer, a workstation, a laptop computer, a mobile computer, a Smartphone, a tablet computer, a cell phone, a personal digital assistant, a gaming system, a kiosk, any other device that utilizes a graphical user interface, including office equipment, automobiles, industrial equipment, and/or a television, for example. In one embodiment, for example, the computing system 150 comprises a tablet computer that provides a user interface responsive to contact with a human hand/finger or stylus.

The computing system 150 may run an off-the-shelf operating system 154 such as a Windows, Linux, MacOS, Android, iOS, or other. The computing system 150 may also run a more specialized operating system which may be designed for the specific tasks performed by the computing system 150.

The computing system 150 may include one or more hardware computing processors 152. The computer processors 152 may include central processing units (CPUs) and may further include dedicated processors such as graphics processor chips, or other specialized processors. The processors generally are used to execute computer instructions based on the software modules 151 to cause the computing device to perform operations as specified by the modules 151.

The various software modules 151 (or simply “modules 151”) may be provided on a computer readable medium, such as a compact disc, digital video disc, flash drive, or any other tangible medium. Such software code may be stored, partially or fully, on a memory device of the executing computing device for execution by the computing device. The application modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. For example, modules may include software code written in a programming language, such as, for example, Java, JavaScript, ActionScript, Visual Basic, HTML, C, C++, or C#. While “modules” are generally discussed herein with reference to software, any modules may alternatively be represented in hardware or firmware. Generally, the modules described herein refer to logical modules that may be combined with other modules or divided into sub-modules despite their physical organization or storage.

The computing system 150 may also include memory 153. The memory 153 may include volatile data storage such as RAM or SDRAM. The memory 153 may also include more permanent forms of storage such as a hard disk drive, a flash disk, flash memory, a solid state drive, or some other type of non-volatile storage.

The computing system 150 may also include or be interfaced to one or more display devices 155 that provide information to the users. A display device 155 may provide for the presentation of GUIs, application software data, and multimedia presentations, for example. Display devices 155 may include a video display, such as one or more high-resolution computer monitors, or a display device integrated into or attached to a laptop computer, handheld computer, Smartphone, computer tablet device, or medical scanner. In other embodiments, the display device 155 may include an LCD, OLED, or other thin screen display surface, a monitor, television, projector, a display integrated into wearable glasses, such as a virtual reality or augmented reality headset, or any other device that visually depicts user interfaces and data to viewers.

The computing system 150 may also include or be interfaced to one or more input devices 156 which receive input from users, such as a keyboard, trackball, mouse, 3D mouse, drawing tablet, joystick, game controller, touch screen (e.g., capacitive or resistive touch screen), touchpad, accelerometer, video camera and/or microphone.

The computing system 150 may also include one or more interfaces 157 which allow information exchange between computing system 150 and other computers and input/output devices using systems such as Ethernet, Wi-Fi, Bluetooth, as well as other wired and wireless data communications techniques.

The modules of the computing system 150 may be connected using a standard based bus system. The functionality provided for in the components and modules of computing system 150 may be combined into fewer components and modules or further separated into additional components and modules.

In the example of FIG. 1, the computing system 150 is connected to a computer network 160, which allows communications with various other devices, both local and remote. The computer network 160 may take various forms. It may be a wired network or a wireless network, or it may be some combination of both. The computer network 160 may be a single computer network, or it may be a combination or collection of different networks and network protocols. For example, the computer network 160 may include one or more local area networks (LAN), wide area networks (WAN), personal area networks (PAN), cellular or data networks, and/or the Internet.

Various devices and subsystems may be connected to the network 160. For example, one or more medical imaging device that generate images associated with a patient in various formats, such as Computed Tomography (“CT”), magnetic resonance imaging (“MRI”), Ultrasound (“US”), (X-Ray) (“XR”), Positron emission tomography (“PET”), Nuclear Medicine (“NM”), Fluoroscopy (“FL”), photographs, illustrations and/or any other type of image. These devices may be used to acquire images from patients, and may share the acquired images with other devices on the network 160. Medical images may be stored in any format, such as an open source format or a proprietary format. A common format for image storage in the PACS system is the Digital Imaging and Communications in Medicine (DICOM) format.

In the example of FIG. 1, the computing system 150 is configured to execute one or more of a speech recognition module 165, Natural Language Processing (“NLP”) module 170, and/or composite image generation module 180. In some embodiments, the modules 165, 170, 180 are stored partially or fully in the software modules 151 of the system 150. In some implementations, one or more of the modules 165, 170, 180 may be stored remote from the computing system 150, such as on another device that is accessible via a local or wide area network (e.g., via network 160).

The modules 165, 170, 180 may each include one or more machine learning models that are generally usable to evaluate input data to provide some output data. As noted above with reference to software modules 151, the modules may comprise various formats and types of code or other computer instructions, including software that does or does not employ machine learning. In one implementation, the modules are accessed via the network 160 and applied to various formats of data at the computing system 150. In some embodiments, the various modules 165, 170, 180, may be executed remote from the computing system 150, such as at a cloud device (e.g., one or more servers that is accessible view the Internet) dedicated for evaluation of the particular module (e.g., including the machine learning model(s) in the particular module). Thus, even if a particular computerized processes is described herein as being performed by a particular computing device (e.g., the computing system 150), the processes may be performed partially or fully by other devices.

In some embodiments, the modules 165, 170, and/or 180 may include a model execution device configured to evaluate one or more models of the module based on the received input data. For example, the speech recognition module 165 may receive an audio stream input (either prerecorded or real-time, live audio stream) and provide a textual interpretation of speech in the audio stream as an output. This module may or may not employ a machine learning algorithm. The NLP module 170 may receive a textual input (e.g., text in a medical report or text output directly from the speech recognition module 165) and provide an output indicating various attributes of the textual input. As discussed further herein, an NLP model may be configured to identify anatomical features and related characteristics of the anatomical feature (e.g., a state of the anatomical feature) that is described in a textual input. A composite image generation module 180 may receive a textual input, such as from the output of an NLP model, indicating an anatomical feature and condition (e.g., supraspinatus, abnormal or supraspinatus, 1 cm full thickness tear located 2 cm from the tendon insertion on the greater tuberosity of the humeral head), and select a corresponding image from a library of images and/or generate a graphical representation associated with the anatomical feature and condition. Each of these modules may include various artificial intelligence (“AI”) algorithms or non-AI programs to generate the corresponding output.

In the example of FIG. 1, an image library 190 is in communication with the network 160 and, thus, may communicate with one or more of the computing system 150 and/or the modules 165, 170, 180. As noted above, images of various types may be selected based on descriptions of patient anatomy by a physician, for example, and combined in some manner to generate a composite (or “dynamic”) image that is representative of the anatomical features and states of those anatomical features. Depending on the embodiment, the types of images may include one or more of illustrations 182 (e.g., a drawing, painting, cartoon, generated manually or digitally by an artist), photographs 184 (e.g., captured by a camera or other optical sensor), medical images 186 (e.g., obtained by medical imaging equipment, such as x-ray, CT, MRI, ultrasound, nuclear medicine, etc.), artificial intelligence (“AI”) images 188 (e.g., images created by artificial intelligence), composite images 192 (e.g., images created as composites of multiple other images with certain anatomical features in a normal state and certain anatomical features in an abnormal state), and/or any other type of image. In some implementations, the composite images are those generated by the composite image generation module 180, which increases size of the library over time and may reduce the frequency of needing to generate new composite images as more combinations of anatomical features and characteristics are already stored in the image library 190.

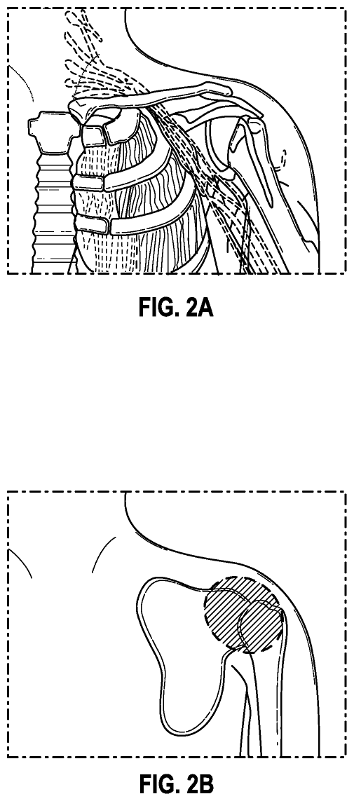

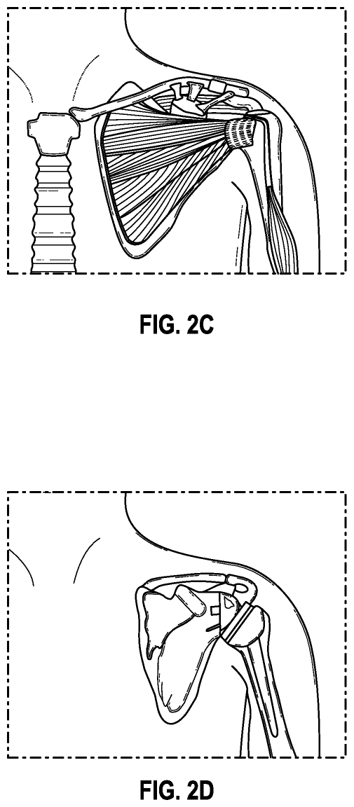

FIGS. 2A-2D illustrate examples of different types of images that are each representative of a same anatomic structure, each with different visual characteristics and variations in level of detail. In this example, FIG. 2A is a detailed cut-away image, FIG. 2B is a simplified line art representation, FIG. 2C is a detailed overlay of underlaying anatomical features, and FIG. 2D is sketch. The types of images that may be used in generating a composite image are not limited to these examples, and include any other style, type, complexity, etc. of image. In addition, the examples in FIG. 2 are created to illustrate the intention of this invention and are not intended to be anatomically precise or precisely representative or set limitations upon the type of images, whether 2D or 3D (e.g., volumetric image), to be used in products that are based in the invention described herein.

In some embodiments, the image library 190 may include multiple data stores, either co-located and/or remotely located. For example, the illustrations 182 may be stored on a first server or system at a first location (e.g., a third-party cloud server of medical illustrations), while medical images 186 are stored at a second server or system at a second location (e.g., a hospital PACS system).

While the description below provides examples with reference to medical imaging management and reporting systems, the systems and methods discussed herein are not restricted to the medical field. In one example implementation that is discussed for purposed of illustration, an expert reading physician may interpret an MRI exam of the shoulder (or any other image) comprising hundreds or more images. In one embodiment, the physician dictates the observed normal and abnormal (e.g., pathological) findings. In some embodiments, the reading physician inputs only the abnormal findings that are then included in a pre-prepared normal report template, so that the abnormalities are included in the report and the appropriate normal findings from the normal report template remain present. In some embodiments, the physician inputs abnormal findings and optionally normal findings using a form that enables selection of findings using dropdown menus, radio buttons, checkboxes, software buttons, diagrams and/or any other input controls. In other implementations, descriptions of the medical images may be acquired in any other manner from a reading physician or other viewer. In some cases, the input may be derived for image analytics using AI.

### Example Technical Improvements

The systems and methods discussed herein provide a more effective and efficient means of generating, selecting, and/or presenting images (e.g., composite images) based on combinations of features from multiple images from a library. The technical features and advantages may include one or more of the features discussed below.

As discussed further herein, the computing system 150 may be configured to generate and display composite images that are a composite of multiple images of various types that are stored in the library 190 (and/or other sources). The various images in the image library may each be associated with metadata indicating characteristics of the corresponding image, where the characteristics may be automatically detected in the images (e.g., by artificial intelligence analysis of image features) and/or manually provided by an interpreter of the image (e.g., a radiologist). For example, the image metadata may indicate an anatomical feature(s) depicted in the image (e.g., a particular muscle, tendon, ligament, bone, etc.) and one or more characteristics (e.g., a binary indication of normal or abnormal and/or some more quantitative or qualitative characteristic) of the anatomical feature(s). Thus, the metadata associated with an image may include various levels of detail regarding each of one or more anatomical feature in the image, such as one or more quantitative characteristics (e.g., a measurement or dimension), an indication of severity of a condition, an indication of the stage of the condition, or any other type of characteristic. In some implementations, the image library 190 may include separate images for each anatomical feature, such as separate images for each of multiple muscles, tendons, ligaments, bones, etc.

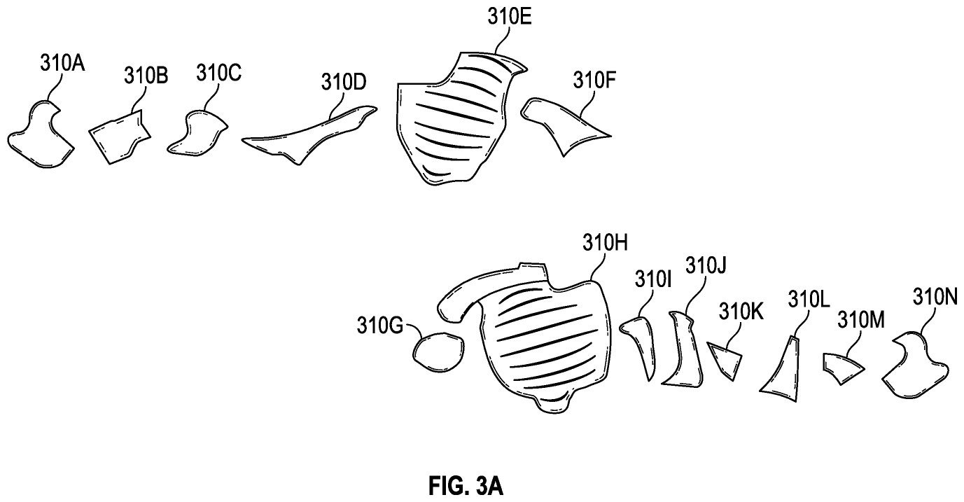

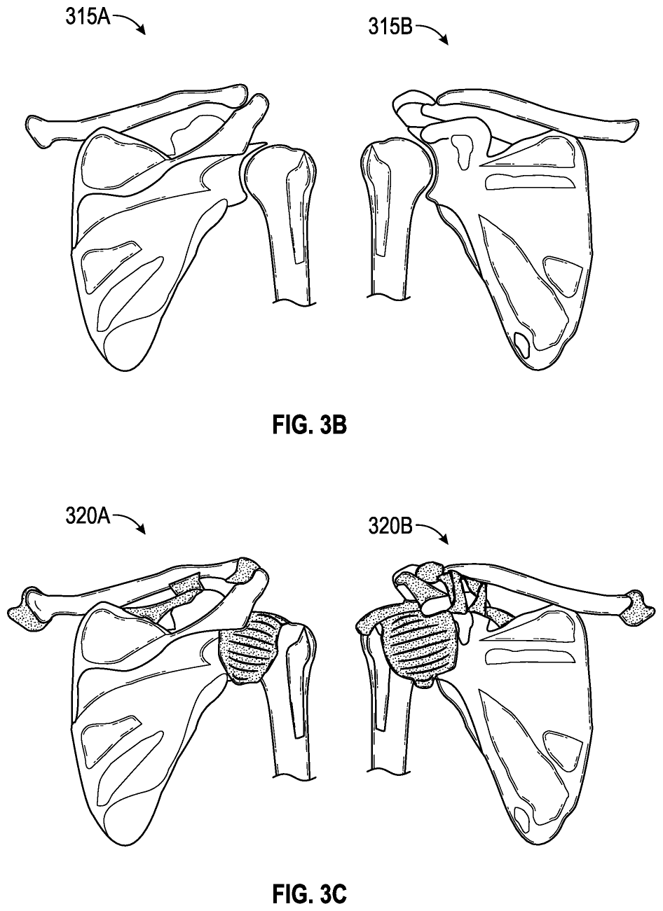

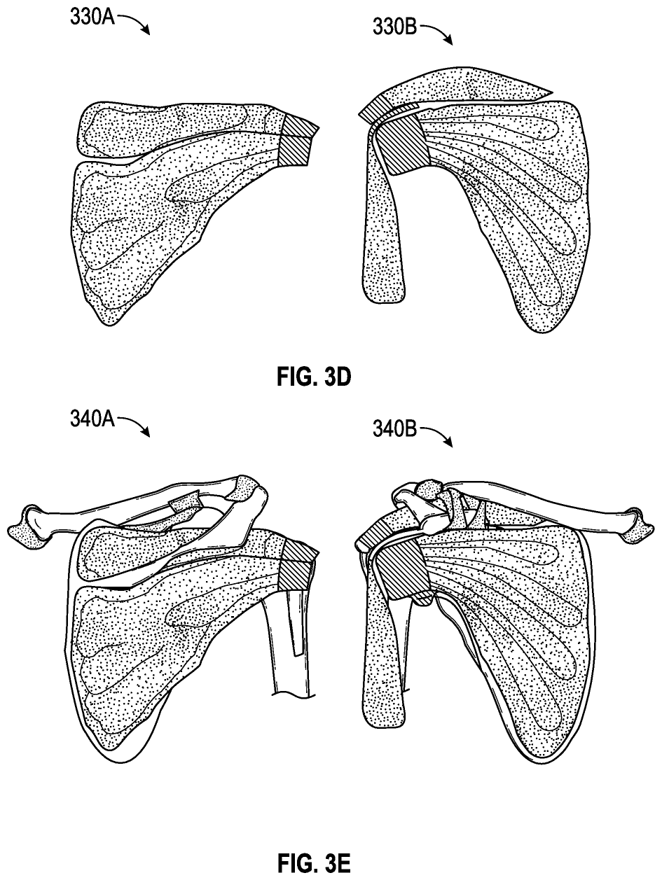

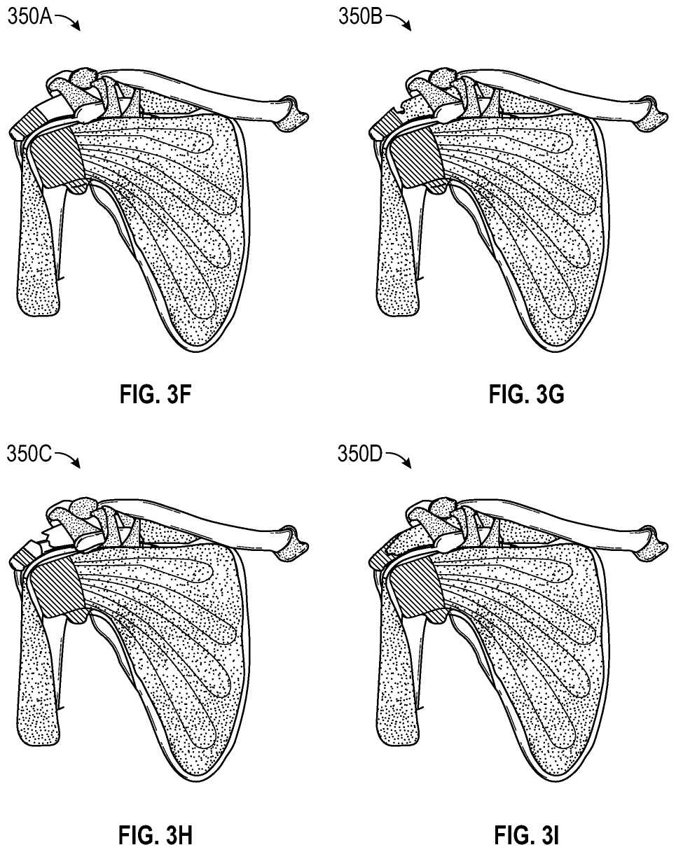

FIG. 3A illustrates several example images of different anatomical features 310 (including features 310A-310N) associated with shoulders (also referred to herein as “feature images.” Each of the features 310 may be stored as a separate image file, such as in the image library 190, along with metadata indicating the particular anatomical feature and characteristic of the feature. For example, feature 310E may be associated with metadata indicating that the feature 310E is the posterior shoulder capsule with a normal state. Additionally, the metadata may indicate other characteristics of the anatomical feature or image, such as whether the anatomical feature is normal or abnormal, quantitative characteristic, qualitative characteristic, image type, size, quality, and/or any other information. Thus, the image library may include multiple images of the bones, muscles, ligaments, cartilage, blood vessels, or other structures that have different characteristics. For example, the image feature 310E may include, or be associated with, metadata indicating that the image is of a posterior shoulder capsule (anatomical feature), normal state (state of anatomical feature), shaded line art (type of image), 320×320 (size of image). Thus, the image library may include multiple images of a same anatomical feature, but with different characteristics.

In some implementations, the image library may store multiple images of the posterior ligamentous shoulder capsule, including a first with the normal state (e.g., image feature 310E), and a second with an abnormal state. Similarly, separate images may be stored for multiple different types of images, such as a first image of a ligament xyz that is shaded line art (e.g., image feature 310E), a second image of the ligament xyz that is a sketch, and a third image of the ligament xyz that is an icon. Thus, for a particular anatomical feature, many different images of the anatomical feature having different combinations of characteristics may be stored in the image library 190. As a specific example, an image library may include multiple images that depict a supraspinatus tendon that range from normal to a complete tear with muscle atrophy and retraction, such as in a series of 10 images. As another example, the library may include multiple images depicting various pathological appearances of the glenoid labrum. These multiple images are then selectable by the computing system to generate a composite image that represents the current state of anatomical feature, such as the shoulder.

FIG. 3B illustrates an example of images 315A and 315B showing a front and back view of bones associated with a shoulder. In some embodiments, the shoulder images 315 may each include a single image (e.g., a single image 315 may show all of the bones, such as if all of the bones are normal) or may be a composite of multiple images associated with the different bones (e.g., multiple individual bones may be selected from images in the image library, such as to include one or more bone images illustrating an abnormal state).

FIG. 3C illustrates an example composite image that is generated by selection of images depicting anatomical features 310A-310N from an image library, which are then overlaid on (or otherwise merged, blended, or combined) with the underlying bone images 315A and 315B. These multiple images of ligaments may be selected based on processing of an already generated medical imaging report or may be selected as part of an iterative process wherein a user provides incremental description of patient anatomy and the computing system then selects a corresponding one or more images to be included in a composite image. In this example, images showing multiple ligaments are combined with one or more bone images to form the illustrated composite images 320A and 320B. In some embodiments, anatomical features that are abnormal may be illustrated with a coloring, texture, or other visual appearance, that distinguishes from anatomical features that are normal.

FIG. 3D illustrates an example composite image of muscles and some tendons. The muscles may each comprise one or more muscle images, such as to indicate any anatomical features that are indicated as abnormal by the user and/or from text that is parsed from a medical report.

FIG. 3E is an example composite image showing combinations of anatomical features of various types (e.g., bones, ligaments, muscles) all combined into a composite medical image 340A and 340B. In some embodiments, the user interface may include options to allow the user to remove one or more of the anatomical features, such as one or more of the muscles, to show ligaments and muscles that are behind the removed anatomical feature.

FIGS. 3F-3I each illustrate a composite medical image, each with different features images associated with different states of the supraspinatus tendon and/or supraspinatus muscle included in the composite medical images. For example, the composite images 350A-3501 may be generated based on identification of different characteristics of the supraspinatus tendon and/or muscle included in the text description from a reading physician, information extracted from a report, and/or other analysis of a medical image or medical data of a particular patient. In this example, the composite image 350A shows the supraspinatus tendon and muscle in a normal state. The composite image 350A may be generated using all normal or template feature images and/or may be a pre-generated image of the all-normal features. The composite image 350B is a composite image illustrating a partial tear along the bursal margin of the supraspinatus tendon adjacent to the musculotendinous junction. Thus, the feature images used to generate the composite image 350B may include a feature image of the supraspinatus tendon that indicates the partial tear in combination with feature images of other anatomical features in a normal state (as illustrated in the composite image 350B). The composite image 350C illustrates a complete tear of the supraspinatus tendon with mild retraction of the supraspinatus muscle. Thus, the feature images used to generate the composite image 350C may include a feature image of the supraspinatus tendon with a complete tear and a featured image of the supraspinatus muscle indicating the mild retraction. The composite image 350D illustrates a complete avulsion of the supraspinatus tendon from its insertion on the greater tuberosity of the humeral head with moderate retraction of the supraspinatus muscle. Thus, feature images of the supraspinatus tendon, supraspinatus muscle, and/or other anatomical features associated with this condition, may be selected and used in combination with other feature images that show normal anatomical features.

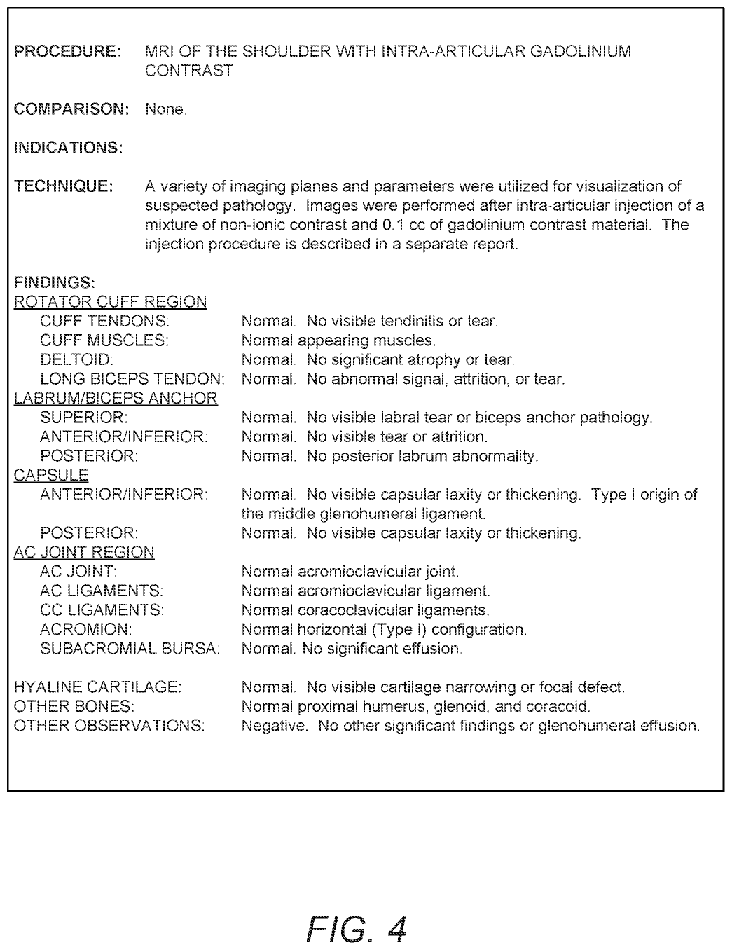

FIG. 4 is an example imaging report 410 that may be processed to generate a composite image. In this example, an itemized list of anatomical findings are shown along with a description of pertinent normal findings. Using the itemized list may enable the system to depict the items that are listed. The user and/or an AI image analysis system may determine with of the itemized findings are normal vs. abnormal or describe either a normal or abnormal finding. Thus, the computing system may identify those abnormal items and identify images from an image library that show each of the anatomical features in a normal or abnormal state. The imaging report 410 may be part of a user interface displayed to a user on a computing device, which may allow the user to view and manipulate the report, as well as to navigate to related information associated with items in the report, such as via link to a medical image associated with an abnormal finding included in the report.

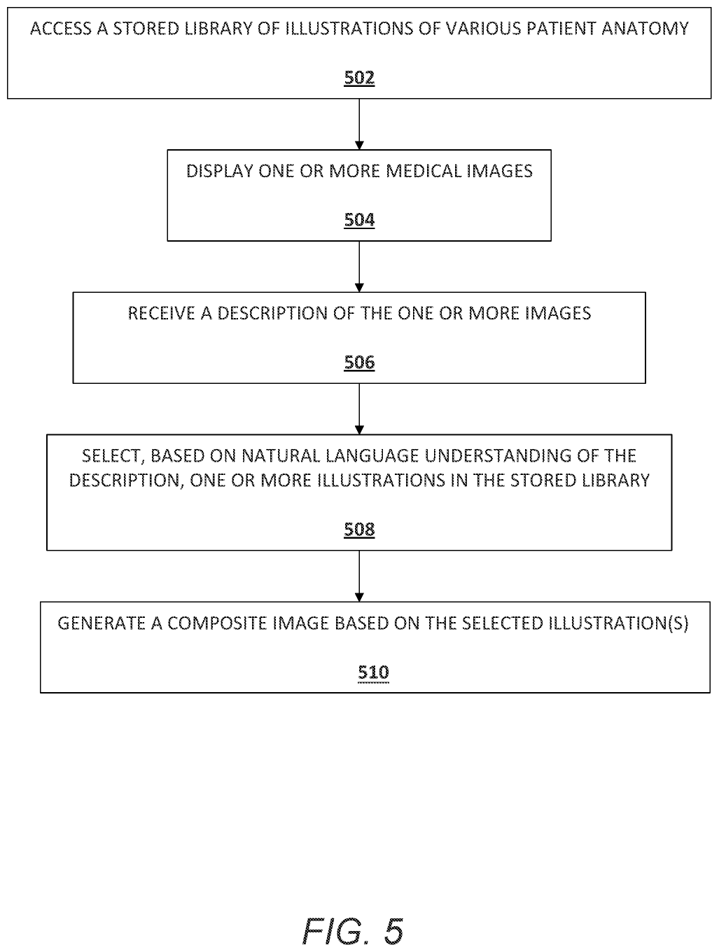

FIG. 5 is a flowchart illustrating one example of a method that may be performed to generate a composite image based on one or more images matching descriptions of patient conditions. In the example of FIG. 5, at block 502 the system access a stored library of illustrations of various patient anatomy, such as images of specific anatomical features in various states. At block 504, the system displays one or more medical images, such as a medical image that the viewer will interpret for possible abnormalities. At block 506, the system receives, from a viewer of the one or more medical images, a description of the one or more images. The description may include one or more indications of normal and abnormal anatomical features. At block 508, the system selects, based on natural language understanding of the description, one or more illustrations in the stored library. At block 510, the system generates a composite image based on the selected one or more illustrations associated with the description.

In some embodiments, other inputs may be provided to initiate updates to a composite image with further images from the image library. For example, a reading physician may input text (e.g., providing further description of the patient anatomy that can be mapped to characteristics in metadata of additional library images) or may navigate through and select particular images. In some embodiments, the composite image generation module receives an indication of whether the user accepts the composite image (e.g., an indication of whether the composite image accurately represents the information in the report and/or provided by the user) and executes a self-learning process to optimize one or more models of the modules 165, 170, or 180.

In the report generation process, the computing device may not require the user to input the anatomical location being described, but instead rely on image segmentation to understand the location. For example, a reading physician may place a cursor over the supraspinatus tendon in an imaging exam, dictate “Complete tear with muscle atrophy” and using image segmentation, the system will understand to select a diagram that includes a complete tear of the supraspinatus tendon with muscle atrophy.

In some embodiments, once a composite image is created, an AI algorithm, such as a generative adversarial network, might further modify the image to match the description. Incrementally adding to a composite image, e.g., by morphing additional illustrations as they are selected by a user, may not only make the process more accurate but also save time, since descriptions might be brief . . . for example, “Buford complex.”

In some embodiments, matching images in the library to images of a patient may add metadata to the composite image. The metadata might be, for example, the image scale or precise position information. In medical imaging, the metadata may be stored in a DICOM metafile for each image, often called the DICOM header. Matching images could result in a common DICOM Frame of Reference, also stored in the DICOM header. As a result, if a user clicks on a particular location in a medical image, that location can be specified in the composite image and vice versa. Therefore, a composite image could be used by the user to help locate a finding on the medical imaging exam. For example, a user could point at a tear of a tendon on the composite image, whereupon the system would show the user the location of the tear on multiplanar MR images of the patient.

In some embodiments, the system may be configured to generate a report by selecting the proper image templates and building a composite image. If the user builds the proper composite image, the text could be generated by the system, with or without the additional pertinent negative findings in the template.

In some embodiments, report language may be automatically generated and/or modified based on the final composite image (e.g., a composite image including multiple images from the image library). For example, a reading physician may input findings in various sequences or may approximate the language description when creating the composite image, such as through multiple real-time updates of the composite image as additional library images matching additional characteristics of the patient are included in the composite image. The system may then use the final composite image to create a written report that uses more precise language, that re-orders the findings, that links each description to specific annotated portions of the composite image, and/or that adds referenced classification system descriptions to the findings. For example, based on a composite image, the system might add description that includes a classification for the appearance of a specific anatomical feature (e.g., the anterior capsule). In another example, if the composite image applies to a Chest CT exam, the system may add a description of the tumor stage using a classification system, such as TNM staging system, and may even auto-label the basis for the TNM classification result.

In some embodiments, the system may provide one or more of the following functions:
Findings section of the report may be replaced or supplemented with one or more labeled illustrations with captions describing the findings. The image library may be configurable and tied to the preferences of the user organization, individual user, or user subgroup.
Other options described above, such as whether the final composite image is used to create the written report or whether or which referenced classification system for the finding is added to the report may be configurable and tied to the preferences of the user organization, individual user, or user subgroup.
The images in the library may be linked to exam types (e.g., based on metadata of particular images) or may be automatically selected based on the input description of the findings (e.g., from the NLP model) matching other metadata of the images.
The system may use color coding, grayscale coding or other such methods to distinctly present normal and pathological portions of the composite.
Multiple composite images may be created showing the anatomy from various vantage points or different image types or styles.
Since the composite images reflect discrete information, analog reports may be transformed into discrete data elements that can be used for various purposes, such as teaching, quality assessment, research, public health tracking, etc.
The composite images can also be used to compare exams, such as illustrating the progression or regression of disease. This can substantially speed the production of a report. For example, a reading physician may edit just one portion of a prior exam composite image to indicate that the current exam is unchanged from the prior exam except for that one element. The system may then generate a text report that includes all of the normal and pathologic findings from the prior report except for the edited area, where the current report is updated to reflect the change.
The system may use a text description to create a composite image that reflects a prior surgical procedure or surgical implant. For example, a reading physician may describe a post-operative appearance of the stomach and small intestine on a CT or MRI. Based on the description, the system may select an illustration that is labeled Billroth II or Roux-en-Y Gastric Bypass. Thus, the reading physician need not memorize the name of the surgical procedure or the name of various implanted devices. Alternatively, the reading physician may provide the name of a surgical procedure based on the patient's history in order to help create the proper composite image, such as by dictating Roux-en-Y Gastric Bypass.
The system may segment an implanted device to identify it by name for the purpose of creating a composite image.

The systems and methods discussed herein may provide various technical features and/or advantages over existing technology, such as though combination of a speech recognition, natural language processing, and composite image generation to create composite images, such as composite and/or generative images, illustrating reported findings. Additionally, the systems and methods discussed herein may advantageously:
update an image library and/or machine learning models (e.g., models of modules 165, 170, 180 of FIG. 1) based on use of the system.
use a generated composite image to create corresponding text, such as may be included in a report.
add referenceable finding classification systems to the report based on the composite image.
use image segmentation with the various other aspects to speed and improve the accuracy of creating composite images.
replace the findings of a report with one or more labeled composite images with captions.
The described technology may advantageously improve the speed and accuracy of creating composite images as well as the quality of the report of imaging findings.

### Additional Implementation Details and Embodiments

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums).

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

As described above, in various embodiments certain functionality may be accessible by a user through a web-based viewer (such as a web browser), or other suitable software program. In such implementations, the user interface may be generated by a server computing system and transmitted to a web browser of the user (e.g., running on the user's computing system). Alternatively, data (e.g., user interface data) necessary for generating the user interface may be provided by the server computing system to the browser, where the user interface may be generated (e.g., the user interface data may be executed by a browser accessing a web service and may be configured to render the user interfaces based on the user interface data). The user may then interact with the user interface through the web-browser. User interfaces of certain implementations may be accessible through one or more dedicated software applications. In certain embodiments, one or more of the computing devices and/or systems of the disclosure may include mobile computing devices, and user interfaces may be accessible through such mobile computing devices (for example, smartphones and/or tablets).

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain embodiments include, while other embodiments may not include, certain features, elements, and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain embodiments require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a general purpose computer comprising one or more processors should not be interpreted as excluding other computer components, and may possibly include such components as memory, input/output devices, and/or network interfaces, among others.

While the above detailed description has shown, described, and pointed out novel features as applied to various embodiments, it may be understood that various omissions, substitutions, and changes in the form and details of the devices or processes illustrated may be made without departing from the spirit of the disclosure. As may be recognized, certain embodiments of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A computing system comprising:
a hardware computer processor;
a non-transitory computer readable medium having software instructions stored thereon, the software instructions executable by the hardware computer processor to cause the computing system to perform operations comprising:
access a stored library of illustrations of various patient anatomy;
display one or more medical images;
receive, from a viewer of the one or more medical images, a description of the one or more images;
select, based on natural language understanding of the description, one or more illustrations in the stored library; and
generate a composite image based on the selected one or more illustrations associated with the description.

2. The computing system of claim 1, wherein the description of the one or more images is in a medical imaging report.

3. The computing system of claim 1, wherein the description of the one or more images is received via input from the user of the computing system.

4. The computing system of claim 1, wherein the illustrations are indexed based on one or more of an imaging exam type or a report template.

5. The computing system of claim 1, wherein the operations further comprise:
creating a matching DICOM frame of reference between the one or more medical images and the generated composite image.

6. The computing system of claim 1, wherein the composite image is a volumetric composite image.

7. The computing system of claim 6, wherein the operations further comprise:
receiving user input requesting reformatting of the volumetric composite image into a three-dimensional or multiplanar images; and
performing the requested reformatting.

8. The computing system of claim 1, wherein the operations further comprise:
receiving user input selecting a first of the illustrations in the composite image; and
regenerating the composite image without the first of the illustrations, wherein positions of one or more other illustrations become visible in the regenerated composite image.

9. The computing system of claim 1, wherein the operations further comprise:
receiving user input selecting an anatomical feature;
determining one or more portions of the composite image overlapping or obscuring view of the anatomical feature in the composite image; and
regenerating the composite image to at least partially remove the one or more portions of the composite image overlapping or obscuring view of the anatomical feature.

10. The computing system of claim 9, wherein the user input is received by user selection of text in a medical imaging report.

11. The computing system of claim 1, wherein the operations further comprise:
determining a report description associated with the one or more selected illustrations; and
generating at least portions of a report based on the determined report descriptions.

12. A computerized method, performed by a computing system having one or more hardware computer processors and one or more non-transitory computer readable storage device storing software instructions executable by the computing system to perform the computerized method comprising:
(a) determining an anatomical area of a patient;
(b) identifying an image of the anatomical area from a stored library of images of various patient anatomy;
(c) displaying the image;
(d) receiving, from a viewer, a description of a feature of the patient;
(e) determining, based on application of natural language understanding to the description, a characteristic of the patient anatomy;
(f) identifying a feature image in the stored library that is associated with the determined characteristic of the patient anatomy;
(g) generating a composite image based on the image and the feature image;
(h) replacing display of the image with the composite image; and
(i) repeating actions (d) through (h) until no further features of the patient are described by the viewer.

13. The method of claim 12, wherein the characteristic of the patient anatomy indicates whether an anatomical feature is normal or abnormal.

14. The method of claim 12, wherein the characteristic of the patient anatomy indicates a quantitative characteristic of the patient anatomy.

15. The method of claim 12, wherein each of the images in the stored library is associated with metadata indicating characteristics of the image.

16. The method of claim 15, wherein the metadata includes an indication of anatomical area and characteristic of the anatomical area depicted in the corresponding image.

17. The method of claim 12, wherein the image is a photograph of the patient.

18. The method of claim 12, wherein the feature image is an illustration.

19. The method of claim 12, wherein said generating the composite image is performed by a generative artificial intelligence model.

20. The method of claim 12, further comprising: updating the composite image based on an age, gender, height, or weight of the patient.

21. A computerized method, performed by a computing system having one or more hardware computer processors and one or more non-transitory computer readable storage device storing software instructions executable by the computing system to perform the computerized method comprising:
determining an anatomical area of a patient;
parsing a report to identify a plurality of descriptions of the patient;
for each of the identified descriptions in the report:
determining a corresponding anatomical feature;
determining, based on application of natural language understanding to the description, a state of the anatomical feature, wherein the state is either a normal state or an abnormal state;
selecting a feature image in a stored library of images that is associated with the determined anatomical feature having the determined state;
generating a composite image based on each of the selected feature images; and
displaying the composite image.
